(19) United States
(12) Patent Application Publication
(10) Pub. No.: US 2023/0122887 A1
(43) Pub. Date: Apr. 20, 2023

(54) LENGTH-ADJUSTABLE OSSICLE PROSTHESIS

(71) Applicant: MED-EL Elektromedizinische Gerate GmbH, lnnsbruck (AT)

(72) Inventors: Uwe STEINHARDT, Hirrlingen (DE); Jörg HAUSCH, Ofterdingen (DE); Clemens KUEN, Imst (AT); Lukas MAIR, Schwaz (AT)

(21) Appl. No.: 17/915,181

(22) PCT Filed: Mar. 11, 2021
(86) PCT No.: PCT/EP2021/056148

§ 371 Date: Sep. 28, 2022

(30) Foreign Application Priority Data
Mar. 31, 2020 (DE) 10 2020 108 887.6

(51) Int. Cl. A61F 2/18

(52) U.S. Cl. CPC A61F 2/18; A61F 2002/183; A61F 2250/0007

(57) ABSTRACT

A length-adjustable ossicular prosthesis, includes a cylindrical pin serving as a connecting element, which connects a foot element to a head element. The head element is intended to be disposed on an inside of an eardrum, and the foot element is intended to be attached to an anvil in a human ear. The head element is designed as an elliptical ring having an opening, which can be closed by closing a locking mechanism comprising a hook. Two guiding and clamping elements project inwardly from the ring, between which, in an open position, the connecting element is displaceably guided, and which secure the head element non-displaceably and non-rotatably on the connecting element when the ring is closed, and as a result the guiding and clamping elements are tensioned against the connecting element.

The invention relates to a length-adjustable ossicular prosthesis having the features of the preamble of claim 1.

Ossicular prostheses are used to replace one or more ossicles in the human middle ear and transmit vibrations, generated by sound waves, at the eardrum to the oval window of the inner ear, in place of the replaced ossicle or ossicles. The length of an ossicular prosthesis must be individually adapted to the distance to be bridged with respect to the eardrum at the ossicles to be connected, for example the stirrup, or the oval window of the particular patient.

Patent application EP 0 998 884 A2 discloses a length-adjustable ossicular prosthesis comprising a head element, which is disposed on the inside of an eardrum, a foot element, which is fastened to the stirrup, and a pin-shaped shaft, which connects the head element and the foot element. The head element comprises an oval ring, from which arms lead inwardly to a kind of “hub” which has a hole into which a slot leads. The shaft of the known ossicular prosthesis extends through the hole in the “hub” of the head element so that the head element can be displaced on the shaft and, as a result, a distance between the head element and the foot element of the ossicular prosthesis can be adjusted. Pressing the slot together allows the hole to be narrowed, and the head element to be securely clamped on the shaft. The distance between the head element and the foot element is the length of the ossicular prosthesis, and the shaft of the ossicular prosthesis must be cut to this length.

Patent DE 10 2017 115 806 B3 discloses a likewise annular head element for an ossicular prosthesis, which can be displaced and securely clamped on the shaft of an ossicular prosthesis. A ring of the head element comprises an □-shaped arm on the inside thereof, the feet of which transition integrally into the ring at mutually opposing points of the circumference. In a center, the □-shaped arm has a hole for the shaft of the ossicular prosthesis through which a slot extends, which is open on a convex outer side of the □-shaped arm. By elastically pressing the ring together at the feet of the □-shaped arm, the hole can be widened so that the head element can be displaced on the shaft. When the ring is relaxed, the hole thereof is securely clamped on the shaft. The disadvantage of this arrangement is that the shaft can only be displaced while the ring is being elastically pressed together. This makes handling enormously more difficult for the surgeon in order to adjust the length of the ossicular prosthesis.

It is the object of the invention to propose an ossicular prosthesis in which a distance between a head element and a foot element is easily adjustable. The distance between the head element and the foot element is regarded as the length of the ossicular prosthesis.

This object is achieved by the features of claim 1. The dependent claims relate to refinements and advantageous embodiments of the invention.

The ossicular prosthesis according to the invention comprises a head element, a foot element, and a connecting element connecting the head element to the foot element. The head element is intended to be disposed at an inside of an eardrum, and the foot element is intended to be disposed at an ossicle or oval window of the human middle ear, and in particular to be fastened to the stirrup. Eardrum in the present context shall be understood to mean a present, partially present and artificial complete or partially reconstructed eardrum, as well as a partially present eardrum, the remaining portion being reconstructed. The reconstruction encompasses any form of producing a vibratable membrane, for example using body tissue or a synthetic vibrating membrane for mechanical sound transmission. Also encompassed is an arrangement or attachment of the head element at an ossicle, and of the foot element at an ossicle other than the stirrup, or in another spot in a human ear. The ossicular prosthesis in particular comprises a pin as the connecting element.

According to the invention, the head element is designed as a clip or comprises a clip, which in an open or relaxed position can be displaced in a longitudinal direction of the connecting element, and in a tensioned position is securely clamped to the connecting element, so that a distance between the head element and the foot element can be adjusted, and the head element can be secured at the connecting element at the adjusted distance by clamping.

The head element or the clip comprises two or more guiding and clamping elements, which displaceably guide the head element, when the clip is open, at the connecting element in the longitudinal direction of the connecting element and which, when the clip is tensioned, securely clamp the head element to the connecting element. In the process, a clamping force is to be so great that the head element, when the clip is tensioned, does not rotate with respect to the connecting element, nor is displaced, nor moves in another manner with respect to the connecting element, when stresses occur in a human ear. On the other hand, the clamping force is not to be so great that the head element deforms transversely to the axial plane of the ring of the head element or, generally speaking, transversely to the surface thereof, over the long implanted time period.

The head element of the ossicular prosthesis according to the invention furthermore preferably comprises a tensioning element for each guiding and clamping element, which is in particular comparable, for example, to a handle or a lever of pliers, in particular with respect to the function thereof. Using the tensioning elements, the guiding and clamping elements can be tensioned against the connecting element so as to be securely clamped to the connecting element, thereby securing the head element at the connecting element by way of clamping. The tensioning elements are preferably elastic in the tensioning direction so as to apply a tensioning force or clamping force.

So as to hold the head element or the clip in the tensioned position, and thereby secure the head element at the connecting element to prevent displacement and rotation, the head element comprises a locking mechanism that, when closed, holds the tensioning elements in the tensioned position in which the tensioning elements tension the guiding and clamping elements against the connecting element.

When the locking mechanism is open, the head element of the ossicular prosthesis according to the invention can be displaced in the longitudinal direction of the connecting element and, as a result, the distance between the head element and the foot element can be adjusted. When the locking mechanism is open, the head element can be displaced freely, or possibly with friction, in the longitudinal direction of the connecting element, wherein, when the locking mechanism is open, the friction is lower than the clamping force is when the locking mechanism is closed and the clip is tensioned, and preferably is only a fraction of the clamping force. When the locking mechanism is closed and the clip is tensioned, the head element is held at the connecting element, by way of the clamping force, to prevent displacement and rotation. The invention enables a simple adjustment of the distance between the head element and the foot element and allows the head element to be secured at the adjusted distance. “Length-adjustable” within the meaning here shall be understood to mean the adjustment of the distance between the head element and the foot element.

According to one embodiment of the invention, depressions are provided in mutually facing edges or surfaces of the guiding and clamping elements, in or between which the connecting element is accommodated, for displaceably guiding the head element in the longitudinal direction of the connecting element. The depressions are, for example, round or angular, for example circular or V-shaped, and can be interpreted as guides and clamping sections for displaceable guidance in the longitudinal direction of the connecting element when the clip is open, and for secure clamping to or on the connecting element when the clip is tensioned.

In a preferred embodiment of the invention, the head element comprises a ring that is open at a point of the circumference, which can thereby be elastically and/or plastically pressed together in an axial plane of the ring as a result of the open point of the circumference. The guiding and clamping elements project inwardly from the ring so that the distance thereof with respect to one another can be reduced by pressing the ring together, and the guiding and clamping elements can be tensioned against the connecting element so that the head element is secured at the connecting element by clamping. The ring can be closed in the tensioned position by way of the locking mechanism so as to secure the head element at the connecting element. The ring and the guiding and clamping elements form the clip of the head element, wherein the locking mechanism can be interpreted as an integral part of the clip. This embodiment allows the head element to be easily produced from one piece, wherein a one-piece design is a preferred but not mandatory feature of invention.

One embodiment of the invention provides a hook and a mating piece with which the hook can engage for closing the ring, serving as the locking mechanism or as part of the locking mechanism. In one refinement, the locking mechanism comprises two or more hooks on one side of the opening, which are disposed one behind the other along the ring, wherein each of the hooks can engage with the mating piece on the other side of the opening of the ring so that, as a function of the hooks engaged with the mating pieces, the ring is narrower or wider. In this way, the clamping force can be adjusted, or a distance between the guiding and clamping elements can be adapted to the connecting element or to a diameter of the connecting element. The head element can thereby be used for connecting elements having differing diameters. The clamping force can be optimally adjusted for a predefined diameter of the connecting element. In the process, a clamping force is to be so great that the head element, when the clip is tensioned, does not rotate with respect to the connecting element, nor is displaced, nor moves in another manner with respect to the connecting element, when stresses occur in a human ear. However, the clamping force is not to be so great that the head element deforms transversely to the axial plane of the ring of the head element over the long implanted time period.

One embodiment of the invention provides a reduced cross-sectional area at a point of the circumference or in a section of the circumference of the ring of the head element. The reduced cross-sectional area forms a kind of joint about which opposing sections of the circumference of the ring can be pivoted with respect to one another, from the reduced cross-sectional area to the open point of the circumference. The reduced cross-sectional area is in particular located opposite the open point of the circumference, preferably on the inner side of the ring of the head element.

A preferred embodiment of the invention provides application surfaces for applying a closing tool, for closing the ring, to an outer side of the ring. The application surfaces are, for example, depressions in the outer side of the ring, which are shaped so that the closing tool, for example pliers, does not slip in the circumferential direction of the ring when exerting a closing force on the ring, which is to say, pressing ends of the ring together on both sides of the open point of the circumference.

So as to achieve force multiplication, one embodiment of the invention provides that the guiding and clamping elements are disposed at a greater distance with respect to the opening of the ring than with respect to a point of the circumference located opposite the opening. A closing force exerted on the ends, or near the ends, of the ring on both sides of the opening is translated into a larger clamping force in accordance with the lever arms. The lever arms are, on the one hand, the distances between the points at which the closing force is applied and the point of the circumference of the ring of the head element of the ossicular prosthesis which is located opposite the opening, and, on the other hand, the distances between the guiding and clamping elements or the guides and clamping sections and the point of the circumference of the ring located opposite the opening.

All of the features mentioned in the description and/or shown in the drawing can be implemented individually alone, or in any arbitrary combination, in embodiments of the invention. Embodiments of the invention that do not comprise all, but only some of the features of a claim, including of the independent claim, are possible.

The invention will be described hereafter in greater detail based on one exemplary embodiment shown in the drawings. In the drawings:

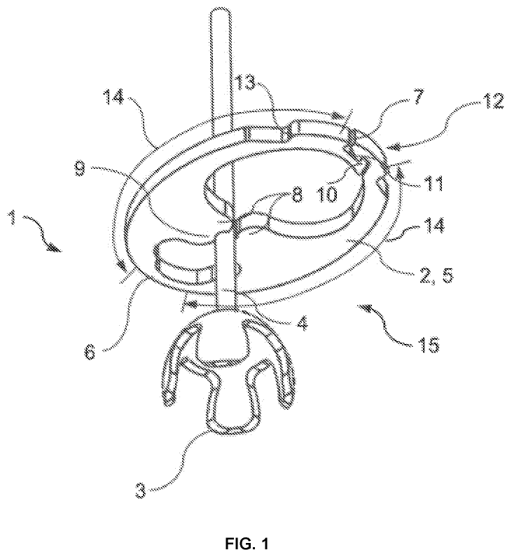

FIG. 1 shows an ossicular prosthesis according to the invention in a perspective illustration; and

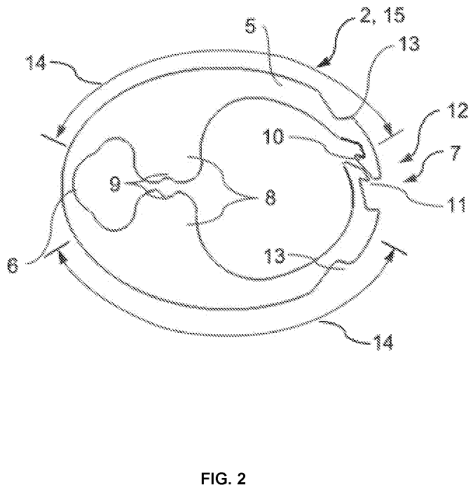

FIG. 2 shows a head element of the ossicular prosthesis in a plan view.

The ossicular prosthesis 1 according to the invention shown in the drawings comprises a head element 2, a foot element 3, and a connecting element 4 connecting the head element 2 and the foot element 4. The ossicular prosthesis 1 is intended to replace one or more ossicles in a human ear. The head element 2 is intended to be disposed on an inside of an eardrum of the ear, and the foot element 3 is intended to be provided at an ossicle, and in particular at a stirrup or an oval window in the middle ear of a person. The ossicular prosthesis 1 transmits vibrations of the eardrum, generated by sound waves, to the stirrup or the element of the ear to which the foot element 3 is attached. Eardrum in the present context shall be understood to mean a present, partially present and artificial complete or partially reconstructed eardrum, as well as a partially present eardrum, the remaining part being reconstructed. The reconstruction encompasses any form of producing a vibratable membrane, for example using body tissue or a synthetic vibrating membrane for mechanical sound transmission.

According to the invention, the head element 2 of the ossicular prosthesis 1 is designed as a clip 15. In the exemplary embodiment, this comprises an approximately elliptic ring 5, which is open at a point of the circumference and, at an opposing point of the circumference or an opposing section of the circumference, is thinner than elsewhere, which is to say has a reduced cross-sectional area 6. In the exemplary embodiment, the open point of the circumference, which is referred to as opening 7 of the ring 5 here, and the reduced cross-sectional area 6 are located opposite one another at the vertices of the ellipse.

Two guiding and clamping elements 8 project inwardly from the ring 5 at points of the circumference that are located opposite one another with respect to a main axis of the ellipsis, which at the mutually facing edges include circular arc-shaped depressions, which are situated on an imaginary circle. The depressions form guides and clamping sections 9 for the connecting element 4. The depressions can also have a different shape, for example a V shape. It is essential that the guides and clamping sections 9 displaceably guide the head element 2, when open, at the connecting element 4 in a longitudinal direction of the connecting element 4, and, when closed or tensioned, secure the head element 2 at the connecting element 4 by clamping to prevent displacement and rotation. Securing to prevent rotation is also conceivable by way of form fit with a connecting element 4 having a non-circular cross-section, on which the head element cannot be rotated (not shown). A clamping force of the closed or tensioned head element 2 is so great that the head element 2 does not move with respect to the connecting element 4 when stresses occur in the human ear. However, the clamping force is not so great that the head element 2 deforms transversely to the axial plane of the ring 5 of the head element 2 over the long implanted time period. A displacement or rotation of the head element 2 by way of a greater force is not precluded.

On one side of the opening 7, the ring 5 comprises a hook 10 on the outer side thereof, and on another side of the opening 7, this comprises a hook on the inner side thereof. The hook on the inner side of the ring 5 forms a mating piece 11 for the hook 10. The hook 10 and the mating piece 11 thereof form a locking mechanism 12 and can engage with one another for closing the ring 5 or the head element 2, designed as a clip 15, of the ossicular prosthesis 1 according to the invention, as is shown in FIG. 1. FIG. 2 shows the head element 2 with an open locking mechanism 12, and an open ring 2 or an open clip 15, the hook 10 not engaging with the mating piece 11.

At a slight distance in the circumferential direction with respect to the hook 10 and the mating piece 11, the ring 10 of the head element 2 has recesses on the outer side thereof, whereby application surfaces 13 for applying a closing tool, which is not shown, are formed. The application surfaces 13 extend parallel to one another and parallel to the main axis of the elliptical ring 5, or the application surfaces 13 extend in a slightly diverging manner in the direction of the opening 7 so that a closing tool applied to the application surfaces 13 does not slip in the circumferential direction of the ring 5.

For closing the ring 5, and thus the clip 15 of the head element 2, the application surfaces 13 are made to approach one another using, for example, pliers (not shown) as a closing tool, which are applied to the application surfaces 13, and as a result, the hook 10 is made to approach the mating piece 11 of the locking mechanism 12 until the hook 10 engages with the mating piece 11, whereby the ring 5 is closed. Closing of the ring 5 in another manner is not precluded.

When the ring 5 is being closed, the reduced cross-sectional area 6 forms a kind of hinge by way of which the arc-shaped sections of the ring 5 between the reduced cross-sectional area 6 and the opening 7 are pivotably connected to one another. The arc-shaped sections of the ring 5 between the reduced cross-sectional area 6 and the opening 7 can also be interpreted as arms, tensioning arms or, generally speaking, as tensioning elements 14 of the clip 15. These are elastic in the exemplary embodiment.

When the ring 5 is being closed, the two guiding and clamping elements 8 and the guides and clamping sections 9 move toward one another and/or are pressed against the connecting element 4, which can also be interpreted as tensioning of the ring 5, of the head element 2 or of the clip 15. The guiding and clamping elements 8 and the guides and clamping sections 9 are located between the reduced cross-sectional area 6, which forms the hinge, and the application surfaces 13 for the closing tool and, as is the case in the exemplary embodiment, are preferably located closer to the reduced cross-sectional surface 6, resulting in a force multiplication: the closing force for pressing the application surfaces 13 together is smaller than a clamping force with which the guides and clamping sections 9 are pressed, which is to say tensioned, against the connecting element 4.

In the exemplary embodiment, the head element 2 is produced in one piece from sheet metal by way of laser cutting. Other production options are not precluded, for example a production from multiple, for example welded pieces and/or a production from wire.

In the shown and described exemplary embodiment of the invention, the connecting element 4 is a cylindrical pin, which passes between the guides and clamping sections 9 of the guiding and clamping elements 8 of the head element 2 and to the one end of which the foot element 3 is fastened, which in the exemplary embodiment has a bell shape, other shapes being possible. The connecting element 4 does not necessarily have to be a cylindrical pin.

When the ring 5 or the head element 2 designed as a clip 15 is open, which is to say the hook 10 is not engaged with the mating piece 11, the head element 2 can be displaced on the connecting element 4 in the longitudinal direction thereof without friction or with friction, and as a result, a distance between the head element 2 and the foot element 3 can be adjusted. Adjusting the distance between the head element 2 and the foot element 3 can also be interpreted as a length adjustment of the ossicular prosthesis 1. By closing the locking mechanism 12, the ring 5, or the head element 2 designed as a clip 15, the guides and clamping sections 9 of the guiding and clamping elements 8 are pressed or tensioned against the connecting element 4 and hold the head element 2 non-displaceably and non-rotatably on the connecting element 4. The closing can also be interpreted as tensioning, and the closed position of the ring 5 or of the clip 15 can be interpreted as a tensioned position.

Finally, the connecting element 4 is cut off on a side of the head element 2 which faces away from the foot element 3 so that the connecting element 4 does not protrude beyond the head element 2 on the side facing away from the foot element 3. Cutting off the connecting element 4 can also be interpreted as cutting the connecting element 4 and the ossicular prosthesis 1 to length.

The elliptical shape of the ring 5 is not mandatory for the invention, and the ring 5 can also have another, preferably rounded, but potentially angular, shape. The ring 5 is also not mandatory for the invention in every case; what is necessary is that the head element 2 is designed as a clip 15, comprising tensioning arms or similar tensioning elements 14, from which the guiding and clamping elements 8 project in the direction of the connecting element 4 so that the guides and clamping sections 9 thereof can be tensioned against the connecting element 4 so as to securely clamp the head element 2 to the connecting element 4, and the tensioning arms, or generally speaking the tensioning elements 14, can secure the head element 2 in this tensioned position, for example by way of a locking mechanism 12.

1. A length-adjustable ossicular prosthesis, comprising a head element, which is intended to be disposed at an eardrum, a foot element, which is intended to be disposed at an ossicle or oval window of the human middle ear, and a connecting element, which connects the head element and the foot element, characterized: in that the head element comprises a clip including guiding and clamping elements, which, when the clip is open, displaceably guide the head element at the connecting element and, when the clip is tensioned, securely clamp the head element to the connecting element; in that the clip comprises tensioning elements, by way of which the guiding and clamping elements can be tensioned against the connecting element so as to be securely clamped to the connecting element; and in that the clip comprises a locking mechanism that, when closed, holds the tensioning elements in the tensioned position in which the tensioning elements tension the guiding and clamping elements against the connecting element.

2. The length-adjustable ossicular prosthesis according to claim 1, wherein the guiding and clamping elements comprise mutually facing depressions serving as guides and clamping sections in which the connecting element is accommodated.

3. The length-adjustable ossicular prosthesis according to claim 1, wherein the head element comprises a ring that is open at a point of the circumference, from which the guiding and clamping elements project inwardly so that these are tensioned against the connecting element when the ring is pressed together, and that the ring can be closed by way of the locking mechanism in the tensioned position in which the ring tensions the guiding and clamping elements against the connecting element.

4. The length-adjustable ossicular prosthesis according to claim 3, wherein the locking mechanism comprises a hook on one side of an opening of the ring, which can engage with a mating piece on another side of the opening of the ring.

5. The length-adjustable ossicular prosthesis according to claim 4, wherein the locking mechanism comprises two or more hooks behind one another along the ring on one side of an opening, each of the individual hooks being engageable with the mating piece on the other side of the opening of the ring so that a circumference of the ring and/or a distance of the guiding and clamping elements with respect to one another can be adjusted for adaptation to connecting elements having differing diameters.

6. The length-adjustable ossicular prosthesis according to claim 3, wherein the ring has a reduced cross-sectional area at a point of the circumference allowing for more easy pressing together for tensioning the guiding and the clamping elements against the connecting element.

7. The length-adjustable ossicular prosthesis according to claim 3, wherein the ring, on both sides of the opening, comprises application surfaces on the outer side thereof for applying a closing tool for closing the ring, which are shaped so that the applied closing tool does not slip in the circumferential direction of the ring when exerting a closing force on the ring.

8. The length-adjustable ossicular prosthesis according to claim 3, wherein the guiding and clamping elements are disposed at a greater distance with respect to the opening of the ring than with respect to a point of the circumference which is located opposite the opening.

9. The length-adjustable ossicular prosthesis according to claim 1, wherein the foot element is designed to be provided at the stirrup or the oval window of the middle ear of a person.
